(19) United States
(12) Patent Application Publication
(10) Pub. No.: US 2024/0065848 A1
(43) Pub. Date: Feb. 29, 2024

(54) CONFIGURABLE ORTHOPAEDIC IMPLANT SYSTEMS AND METHODS OF REPAIR

(71) Applicant: ARTHREX, INC., Naples, FL (US)

(72) Inventor: James Tyler Clevett, Bonita Springs, FL (US)

(21) Appl. No.: 18/337,489

(22) Filed: Jun. 20, 2023

Related U.S. Application Data
(60) Provisional application No. 63/400,124, filed on Aug. 23, 2022.

(51) Int. Cl. A61F 2/40; A61B 17/16

(52) U.S. Cl. CPC A61F 2/4081; A61B 17/1684; A61F 2/4014; A61B 17/1778

(57) ABSTRACT

This disclosure relates to orthopaedic implant systems and methods for restoring functionality to a bone and/or joint. The implant systems disclosed herein may include one or more components that may be configurable to establish a distance or span between two points of the respective implant system, such as an articular surface and an end of the implant system. A method of installing an orthopaedic implant system is also disclosed.

### CROSS-REFERENCE TO RELATED APPLICATION

This application claims the benefit of U.S. Provisional Application No. 63/400,124 filed on Aug. 23, 2022, which is incorporated herein by reference in its entirety.

### BACKGROUND

This disclosure relates to orthopaedic procedures and, more particularly, to orthopaedic implant systems and methods for repairing bone defects and restoring functionality to a joint.

Joints may be established by at least one long bone. The patient may experience loss caused by fracturing or shattering of the bone due to trauma. A prosthesis may be utilized to restore functionality to the bone.

The prosthesis may be modular and may include one or more components may be selectable from a kit. The surgeon or assistant may assemble the components together to establish the prothesis. The components may include a stem insertable in the bone and may include a head for mounting an articulation member to restore functionality to a joint.

### SUMMARY

This disclosure relates to orthopaedic implant systems and methods for restoring functionality to a joint. The implant systems may include one or more components that may be configurable to set a distance or span of the respective implant system.

A component for an orthopaedic procedure of the present disclosure may include a main body extending along a longitudinal axis between a first end and a second end. The main body may be implantable in tissue. The main body may include a cavity that may extend inwardly from the first end. The main body may have a stepped geometry established by a plurality of tiers including a first tier that may have a first tapered periphery and a second tier that may have a second tapered periphery. The second tapered periphery may be established by circumferential faces of a first set of abutments distributed about the longitudinal axis. The cavity may include a first cavity level and a second cavity level distributed along the longitudinal axis. The first cavity level may include a periphery dimensioned to complement a profile of the first tapered periphery. The second cavity level may include a periphery dimensioned to complement a profile of the second tapered periphery. A set of cavity cutouts may extend outwardly from the periphery of the second cavity level. The set of cavity cutouts may be dimensioned to complement a profile of the first set of abutments.

An orthopaedic implant system of the present disclosure may include a set of components stackable to establish an assembly. Each of the components may include a main body extending along an axis between a first end and a second end. A cavity may extend inwardly from the first end. The main body may have a stepped geometry established by a plurality of tiers distributed along the axis. The plurality of tiers may include a first tier that may have a first tapered periphery and a second tier that may have a second tapered periphery. The second tapered periphery may be established by a first set of abutments. The cavity may include a first cavity level and a second cavity level distributed along the axis. A set of cavity cutouts may extend outwardly from a periphery of the second cavity level. Each adjacent pair of the components may be configurable to establish a collapsed configuration and an extended configuration of a respective portion of the assembly. The collapsed configuration may be established in response to inserting the first set of abutments into the set of cavity cutouts to establish a keyed interface such that a first Morse taper connection may be established between a periphery of the first cavity level and the first tapered periphery, but the extended configuration may be established in response to engagement between the periphery of the second cavity level to establish a second Morse taper connection that may block engagement between the periphery of the first cavity level and the first tapered periphery.

An orthopaedic implant system of the present disclosure may include a set of components stackable to establish an assembly. Each of the components may include a main body extending along an axis between a first end and a second end, and a cavity extending inwardly from the first end. The cavity may be dimensioned to receive the second end of an adjacent one of the components. The main body may have a stepped geometry established by a plurality of tiers distributed along the axis. The plurality of tiers may include a first tier and a second tier. The second tier may include a second periphery established by at least one abutment. The main body may include a circumferential wall having a plurality of recesses extending from the first end. The plurality of recesses may include at least one collapsed recess and at least one extended recess distributed about the axis. The at least one collapsed recess may extend a first distance and the at least one extended recess may extend a second distance that may be less than the first distance relative to the axis. Each adjacent pair of the components may be configurable to establish a collapsed configuration and an extended configuration of a respective portion of the assembly. The collapsed configuration may be established in response to inserting the at least one abutment into the at least one collapsed recess of an adjacent one of the components, but the extended configuration may be established in response to inserting the at least one abutment into the at least one extended recess of the adjacent one of the components.

A method of installing an orthopaedic implant system of the present disclosure may include selecting components from a set of components. Each of the components may include a main body that may have a stepped geometry established by a plurality of tiers distributed along an axis between a first end and a second end. The plurality of tiers may include a first tier that may have a first tapered periphery and a second tier that may have a second tapered periphery. The second tapered periphery may be established by a first set of abutments. The main body may include a cavity having a first cavity level and a second cavity level distributed along the axis. A set of cavity cutouts may extend outwardly from a periphery of the second cavity level. Each adjacent pair of the components may be configurable to establish a collapsed configuration and an extended configuration of a respective portion of an assembly. The collapsed configuration may be established in response inserting the first set of abutments of the adjacent component into the set of cavity cutouts to establish a keyed interface and then engaging a periphery of the first cavity level with the first tapered periphery of the adjacent component to establish a first Morse taper connection, but the extended configuration may be established in response to engagement between the periphery of the second cavity level and the first set of abutments of the adjacent component to establish a second Morse taper connection. The method may include configuring each adjacent pair of the selected components in the collapsed configuration or the extended configuration to establish the respective portion of the assembly.

The present disclosure may include any one or more of the individual features disclosed above and/or below alone or in any combination thereof.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates a perspective view of an exemplary implant system including an assembly arranged in a first configuration.

FIG. 2 illustrates a perspective view of the implant system adjacent to an articular surface and secured to bone, with the assembly arranged in a second configuration.

FIG. 3 illustrates a perspective view of an orthopaedic component.

FIG. 4 illustrates a perspective view of an end of the component of FIG. 3.

FIG. 5 illustrates a side view of the component of FIG. 3.

FIG. 6 illustrates another side view of the component of FIG. 3.

FIG. 7 illustrates an end view of the component of FIG. 5.

FIG. 8 illustrates another end view of the component of FIG. 5.

FIG. 9 illustrates a sectional view of the component taken along line 9-9 of FIG. 6.

FIG. 10 illustrates a sectional view of the component taken along line 10-10 of FIG. 5.

FIG. 11 illustrates a perspective view of a configuration of an assembly of an orthopaedic implant system established by a set of components.

FIG. 12 illustrates a perspective view of another configuration of an assembly of an orthopaedic implant system established by a set of components.

FIG. 13 illustrates a side view of the assembly of FIG. 12.

FIG. 14 illustrates a sectional view of the assembly taken along line 14-14 of FIG. 15.

FIG. 15 illustrates a sectional view of the assembly taken along line 15-15 of FIG. 13.

FIG. 16 illustrates a perspective view of the assembly of FIG. 14.

FIGS. 17-19 illustrate sectional views of the assembly taken along reference planes REF17, REF18 and REF19 of FIG. 12, respectively.

FIG. 20 illustrates a perspective view of another configuration of an assembly of an orthopaedic implant system established by a set of components.

FIG. 21 illustrates a side view of the assembly of FIG. 20.

FIG. 22 illustrates a sectional view of the assembly taken along line 22-22 of FIG. 23.

FIG. 23 illustrates a sectional view of the assembly taken along line 23-23 of FIG. 21.

FIG. 24 illustrates a perspective view of the assembly of FIG. 22.

FIGS. 25-27 illustrate sectional views of the assembly taken along reference planes REF25, REF26 and REF27 of FIG. 20, respectively.

FIG. 28 illustrates an exemplary method of installing an orthopaedic implant system.

### DETAILED DESCRIPTION

This disclosure relates to orthopaedic implant systems and methods for repairing bone defects and restoring functionality to various joints. The disclosed implant systems described herein may include a modular prosthesis that may be adapted to facilitate reconstruction or repair of a bone or joint that may be caused by trauma to the patient. A long bone may have relatively severe proximal bone loss due to fracturing (e.g., 3 part or 4 part fractures) or shattering of the bone due to trauma, or a tumor in the bone, for example. Bone loss may be on the order of approximately one-third of the bone that may indicate a reconstruction procedure. The disclosed implant systems, components and methods may be utilized to restore functionality to long bones, such as a humerus or femur. The disclosed implant systems and components may be utilized to restore functionality to a shoulder joint, including anatomical and reverse shoulder procedures. The implant systems and components may be utilized to restore functionality to other bones and joints, such as a knee, hip, elbow, wrist or ankle.

The surgeon may encounter a range of anatomy and conditions in planning and performing orthopaedic procedures. The anatomy may vary due to differences in the patient population, including sex, age, ethnicity, etc. Defects in the bone and joint and trauma may contribute to the variability and may indicate removal and/or replacement of portions of the bone to restore functionality. Stacking multiple identical segments with Morse taper connections may result in an assembly which may be limited to a fixed total height.

The disclosed implant systems may be configurable to establish a specified dimension, such as a length, height or span between two components of the respective implant system, such as an articulation surface and stem implantable in bone. The disclosed components (e.g., segments) may be arranged in various configurations to establish a specified dimension of the implant system incorporating the components. The surgeon or assistant may select the components from a kit and may configure the components interoperative or preoperatively according to a surgical plan. Individual components may be rotated or “clocked” relative to one another to achieve differing engagement positions or configurations, which may improve the ability to create modular assemblies of various total heights, lengths or other configured dimensioned. The components may be coupled together to establish an assembly, which may be generally in the shape of an elongated shaft. The components establishing the assembly may be the same or may differ.

The components may have a main body including two or more tiered male taper sections that establish a generally stepped shape profile. The male tapers may be sized for press fitting into respective female tapers established along a cavity on an opposite end portion of an adjacent one of the components. A crown of fins interspersed may be established along an end of the main body. Recesses between the fins may be dimensioned to engagement abutments along a periphery of the main body to facilitate stability and rotational alignment for full taper engagement between the selected female taper and respective male taper. Two or more of the recesses may differ in height. The components may be rotationally positioned such that selected recesses engage the abutments along the periphery of the main body and/or a selected male taper engages a respective female taper along the cavity to establish a first (e.g., fully collapsed) configuration or a second (e.g., extended) configuration.

The disclosed components may be utilized to establish a controllably extendable assembly. The components may be stacked to any level of modularity and may be utilized to establish various levels of height or length extension. Utilizing the techniques disclosed herein, the surgeon or assistance may configure the components in a manner that more precisely positions one or more features of the implant system to restore functionality of a bone or joint, which may reduce an amount of native bone or other tissue removed from the patient and which may improve mobility and healing.

A component for an orthopaedic procedure of the present disclosure may include a main body extending along a longitudinal axis between a first end and a second end. The main body may be implantable in tissue. The main body may include a cavity that may extend inwardly from the first end. The main body may have a stepped geometry established by a plurality of tiers including a first tier that may have a first tapered periphery and a second tier that may have a second tapered periphery. The second tapered periphery may be established by circumferential faces of a first set of abutments distributed about the longitudinal axis. The cavity may include a first cavity level and a second cavity level distributed along the longitudinal axis. The first cavity level may include a periphery dimensioned to complement a profile of the first tapered periphery. The second cavity level may include a periphery dimensioned to complement a profile of the second tapered periphery. A set of cavity cutouts may extend outwardly from the periphery of the second cavity level. The set of cavity cutouts may be dimensioned to complement a profile of the first set of abutments.

In implementations, the first tier may include a tapered protrusion establishing the first tapered periphery. The tapered protrusion may extend to the second end.

In implementations, the plurality of tiers may include a third tier having a periphery established by a circumferential wall and a second set of abutments extending axially from the circumferential wall towards the second end. The second set of abutments may be distributed about the longitudinal axis. The circumferential wall may include first and second sets of recesses that may extend from the first end. The first set of recesses may be interspersed with the second set of recesses. The first set of recesses may have a first contour dimensioned to complement a profile of the second set of abutments. The second set of recesses may have a second, different contour dimensioned to complement a lesser portion of the profile of the second set of abutments.

In implementations, the first set of recesses may extend a first distance, and the second set of recesses may extend a second distance that may be less than the first distance relative to the longitudinal axis. A first set of cutouts may be interspersed with the first set of abutments about the longitudinal axis. The first set of recesses may be at least partially circumferentially aligned with the second set of abutments. The second set of recesses may be at least partially circumferentially aligned with the first set of cutouts relative to the longitudinal axis.

In implementations, the first set of abutments may be at least partially circumferentially aligned with the second set of abutments relative to the longitudinal axis.

In implementations, the main body may be dimensioned to interconnect a stem component insertable in bone and an articulation component adapted to mate with an opposed articular surface of an adjacent implant or an adjacent bone.

An orthopaedic implant system of the present disclosure may include a set of components stackable to establish an assembly. Each of the components may include a main body extending along an axis between a first end and a second end. A cavity may extend inwardly from the first end. The main body may have a stepped geometry established by a plurality of tiers distributed along the axis. The plurality of tiers may include a first tier that may have a first tapered periphery and a second tier that may have a second tapered periphery. The second tapered periphery may be established by a first set of abutments. The cavity may include a first cavity level and a second cavity level distributed along the axis. A set of cavity cutouts may extend outwardly from a periphery of the second cavity level. Each adjacent pair of the components may be configurable to establish a collapsed configuration and an extended configuration of a respective portion of the assembly. The collapsed configuration may be established in response to inserting the first set of abutments into the set of cavity cutouts to establish a keyed interface such that a first Morse taper connection may be established between a periphery of the first cavity level and the first tapered periphery, but the extended configuration may be established in response to engagement between the periphery of the second cavity level to establish a second Morse taper connection that may block engagement between the periphery of the first cavity level and the first tapered periphery.

In implementations, the first tapered periphery of the first tier may be established by a tapered protrusion terminating at the second end.

In implementations, circumferentially opposed engagement faces bounding the respective cavity cutouts may be dimensioned to limit relative rotation between the adjacent components in the collapsed configuration, but not in the extended configuration, in response to engagement between the first set of abutments and the respective engagement faces.

In implementations, the plurality of tiers may include a third tier. The third tier may include a second set of abutments that may be circumferentially distributed about the axis and a circumferential wall having a plurality of recesses that may extend from the first end. The plurality of recesses may include a set of extended recesses that may be interspersed with a set of collapsed recesses. The collapsed configuration may be established in response inserting the second set of abutments of the adjacent component into the set of collapsed recesses, but the extended configuration may be established in response to inserting the second set of abutments of the adjacent component into the set of extended recess.

In implementations, the set of collapsed recesses may have a first contour dimensioned to complement a profile of the second set of abutments. The set of extended recesses may have a second, different contour dimensioned to complement a lesser portion of the profile of the second set of abutments.

In implementations, the second set of abutments may be at least partially circumferentially aligned with the set of collapsed recesses, but may be circumferentially offset from the set of extended recesses relative to the axis.

In implementations, a stem component may be insertable into bone. An articulation component may be adapted to mate with an opposed articular surface of an adjacent implant or an adjacent bone. The assembly may interconnect the stem component and the articulation component in an installed position.

An orthopaedic implant system of the present disclosure may include a set of components stackable to establish an assembly. Each of the components may include a main body extending along an axis between a first end and a second end, and a cavity extending inwardly from the first end. The cavity may be dimensioned to receive the second end of an adjacent one of the components. The main body may have a stepped geometry established by a plurality of tiers distributed along the axis. The plurality of tiers may include a first tier and a second tier. The second tier may include a second periphery established by at least one abutment. The main body may include a circumferential wall having a plurality of recesses extending from the first end. The plurality of recesses may include at least one collapsed recess and at least one extended recess distributed about the axis. The at least one collapsed recess may extend a first distance and the at least one extended recess may extend a second distance that may be less than the first distance relative to the axis. Each adjacent pair of the components may be configurable to establish a collapsed configuration and an extended configuration of a respective portion of the assembly. The collapsed configuration may be established in response to inserting the at least one abutment into the at least one collapsed recess of an adjacent one of the components, but the extended configuration may be established in response to inserting the at least one abutment into the at least one extended recess of the adjacent one of the components.

In implementations, the at least one abutment may be axially spaced apart from the set of recesses relative to the axis.

In implementations, the at least one abutment may include a set of abutments distributed about the axis. The at least one collapsed recess may include a set of collapsed recesses and the at least one extended recess may include a set of extended recesses interspersed with the set of collapsed recesses relative to the axis. The collapsed configuration may be established in response to inserting the set of abutments into the set of collapsed recesses of the adjacent one of the components, but the extended configuration may be established in response to inserting the set of abutments into the set of extended recesses of the adjacent one of the components.

In implementations, the set of abutments may be at least partially circumferentially aligned with the set of collapsed recesses relative to the axis.

A method of installing an orthopaedic implant system of the present disclosure may include selecting components from a set of components. Each of the components may include a main body that may have a stepped geometry established by a plurality of tiers distributed along an axis between a first end and a second end. The plurality of tiers may include a first tier that may have a first tapered periphery and a second tier that may have a second tapered periphery. The second tapered periphery may be established by a first set of abutments. The main body may include a cavity having a first cavity level and a second cavity level distributed along the axis. A set of cavity cutouts may extend outwardly from a periphery of the second cavity level. Each adjacent pair of the components may be configurable to establish a collapsed configuration and an extended configuration of a respective portion of an assembly. The collapsed configuration may be established in response inserting the first set of abutments of the adjacent component into the set of cavity cutouts to establish a keyed interface and then engaging a periphery of the first cavity level with the first tapered periphery of the adjacent component to establish a first Morse taper connection, but the extended configuration may be established in response to engagement between the periphery of the second cavity level and the first set of abutments of the adjacent component to establish a second Morse taper connection. The method may include configuring each adjacent pair of the selected components in the collapsed configuration or the extended configuration to establish the respective portion of the assembly.

In implementations, the extended configuration may be established such that the second Morse taper connection may block engagement between the periphery of the first cavity level and the first tapered periphery of the adjacent component.

In implementations, the step of establishing the keyed interface may occur such that circumferentially opposed engagement faces bounding the respective cavity cutouts may limit relative rotation between the adjacent components with respect to the axis.

In implementations, the plurality of tiers may include a third tier. The second tier may interconnect the first and third tiers. The third tier may include at least one abutment that may be established along a periphery of the main body. The main body may include at least one extended recess and at least one collapsed recess extending from the first end. The collapsed configuration may be established in response to inserting the at least one abutment of the adjacent component into the at least one collapsed recess, but the extended configuration may be established in response to inserting the at least one abutment of the adjacent component into the at least one extended recess.

In implementations, the first set of abutments may be at least partially circumferentially aligned with a second set of abutments relative to the axis.

In implementations, the selecting step may include selecting at least three of the components from the set of components. The configuring step may include configuring an adjacent pair of the selected components in the collapsed configuration but configuring another adjacent pair of the selected components in the extended configuration to establish the respective portions of the assembly.

In implementations, the method may include securing one of the selected components of the assembly to a stem component. The method may include inserting the stem component into bone. The method may include securing another one of the selected components of the assembly to an articulation component. The articulation component may be adapted to mate with an opposed articular surface of an adjacent implant or an adjacent bone.

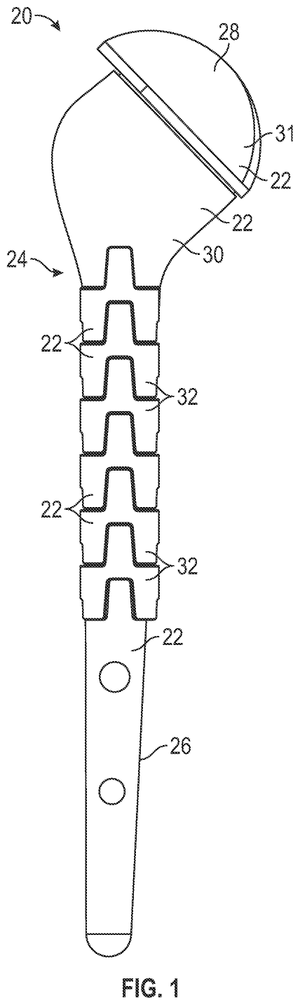

FIG. 1 illustrates an orthopaedic implant system 20. The implant system 20 may be utilized in various orthopaedic procedures to restore functionality to a bone and/or joint, such as a shoulder joint during an anatomical or reverse shoulder arthroplasty. The implant system 20 may be utilized to restore functionality to one or more bones in a fractured or shattered condition. The implant system 20 may be utilized to restore functionality to long bones and other bones such as a humerus or femur. The implant system 20 may be utilized to restore functionality to other bones and joints, such as a knee, hip, elbow, wrist or ankle.

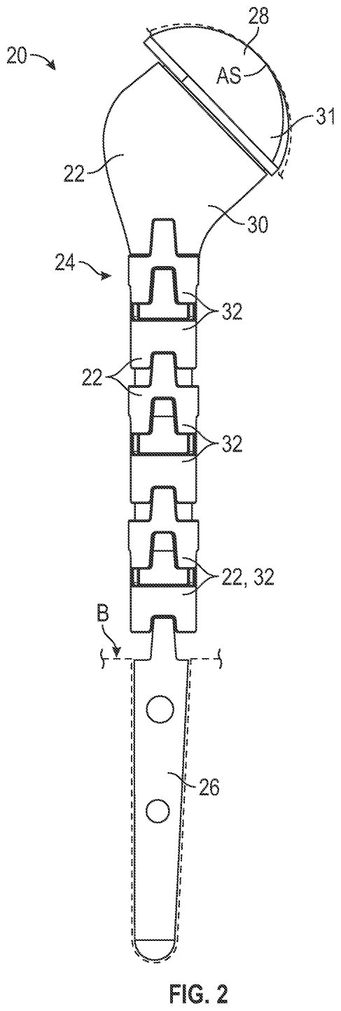

The implant system 20 can include one or more implant components 22. Two or more of the components 22 may be releasably secured to each other to establish an assembly 24. Two or more of the components 22 may be integrally formed with each other. The implant system 20 may include a set of the components 22. The components 22 may be stackable or otherwise arranged to establish the assembly 24 and/or other portions of the implant system 20. The implant system 20 may include a stem component 26 and articulation component 28. The stem component 26 may be insertable into bone B (shown in dashed lines in FIG. 2) to secure the implant system 20 in the patient.

The articulation component 28 may include an articular surface 31. The articular surface 31 may be adapted to mate with an opposed articular surface AS (shown in dashed lines in FIG. 2). The articular surface AS may be established by an adjacent implant or an adjacent bone. The implant system 20 may include a head component 30. The head component 30 may be adapted to interconnect the articulation component 28 and another portion of the implant system 20, such as the assembly 24 and/or stem component 26. In implementations, the articulation component 28 may be omitted, and the assembly 24 may be configured to interconnect an adjacent pair of stem components 26.

The assembly 24 may be configured to interconnect two or more components 22 of the implant system 20 at a specified distance from each other in an installed position, such as the stem component 26 and the articulation component 28. The specified distance may be determined intraoperatively or preoperatively to establish a position of the system 20 in the patient according to a surgical plan.

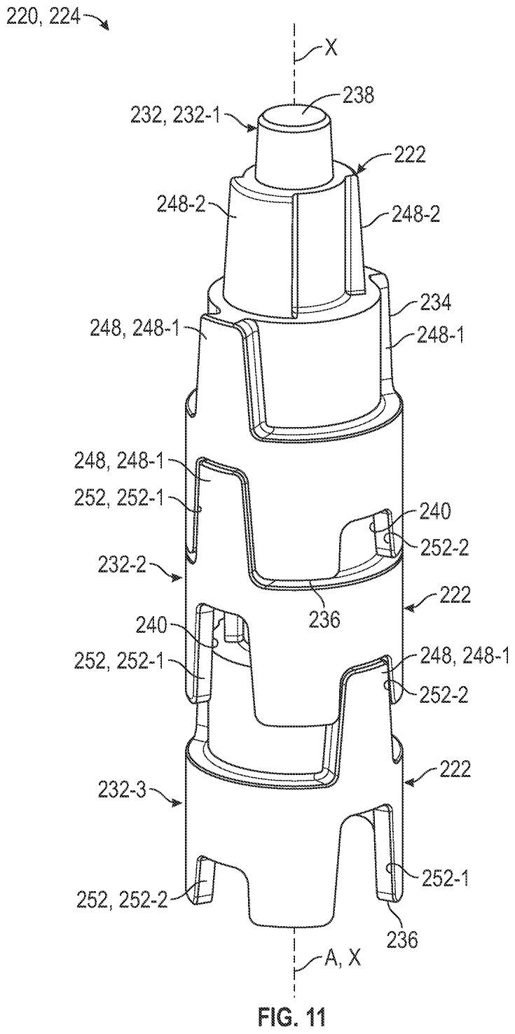

The assembly 24 can be established by a set of components (e.g., links, couplings or segments) 32. The components 32 may be arranged to establish various configurations of the implant system 20. Adjacent pairs of components 32 may be configured or arranged in two or more distinct configurations, including at least a first (e.g., collapsed) configuration and a second (e.g., extended) configuration. In the implementation of FIG. 1, adjacent pairs of the components 32 are arranged to establish a collapsed configuration of respective portions of the assembly 24. In the implementation of FIG. 2, adjacent pairs of the components 32 are arranged to establish an extended configuration of respective portions of the assembly 24. The arrangement of the components 32 establishing the configuration of the implant system 20 of FIG. 2 may have a relatively greater dimension (e.g., height or length) than the arrangement of the components 32 establishing the configuration of the implant system 20 of FIG. 1. One or more pairs of the components 32 may be arranged in the collapsed configuration, and one or more other pairs of the components 32 may be arranged in the extended configuration to establish the assembly 24 (see, e.g., assembly 224 of FIG. 11). The set of components 32 can be common to both of the configurations of FIGS. 1 and 2.

The components 32 may be provided to the surgeon as a kit and may be dimensioned according to various shapes and sizes. The surgeon or assistant may configure and/or reconfigure the components 32 preoperatively and/or intraoperatively according to a surgical plan. Although a total of six of the components 32 is illustrated in configurations of the assembly 24 of FIGS. 1 and 2, it should be appreciated that fewer or more than six of the components 32 may be provided in a set, such as only one or two of the components 32.

Various techniques may be utilized to construct the components 32. Each component 32 may be made of metallic and/or non-metallic materials. Each component 32 may be a monolithic or unitary structure or may include two or more portions mechanically attached or otherwise secured to each other.

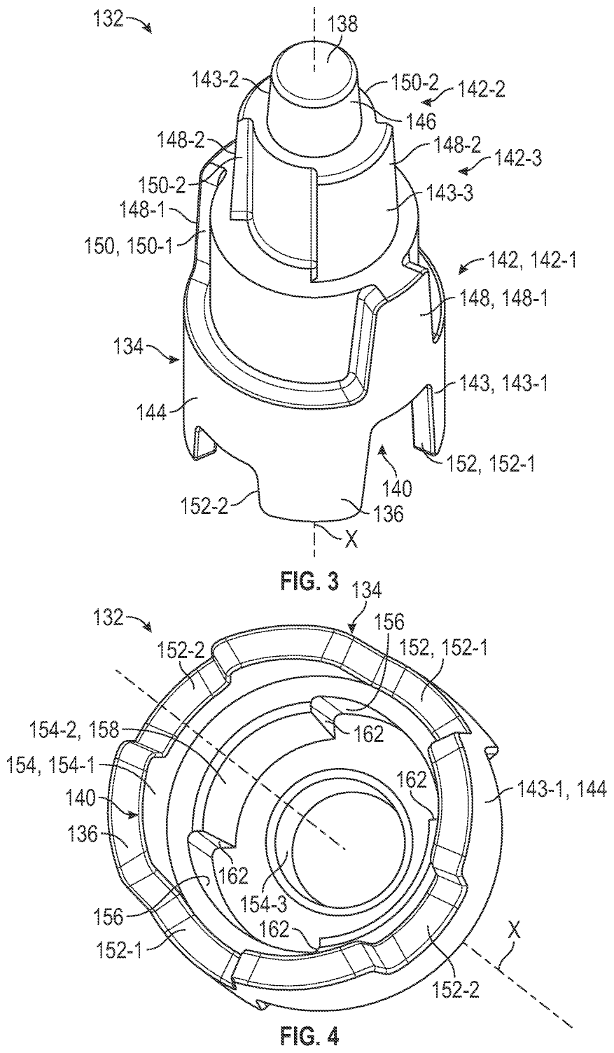

FIGS. 3-10 illustrate an orthopaedic component 132 according to an implementation. The component 132 may be utilized for various orthopaedic procedures, including any of the procedures disclosed herein. In this disclosure, like reference numerals designate like elements where appropriate and reference numerals with the addition of one-hundred or multiples thereof designate modified elements that are understood to incorporate the same features and benefits of the corresponding original elements. The component 132 may be incorporated into any of the implant systems disclosed herein, such as the assembly 24 and/or another portion of the implant system 20. The components 22, 32 of the implant system 20 may incorporate any of the features of the component 132.

Referring to FIG. 3, the component 132 can include a main body 134 extending along an (e.g., longitudinal) axis X between a first end 136 and a second end 138. The main body 134 may be implantable in tissue, such as bone or soft tissue of the patient. In implementations, the main body 134 may be at least partially surrounded by muscle or other soft tissue. In other implementations, the main body 134 may be at positioned at least partially in an intramedullary canal of a long bone. The main body 134 may be dimensioned to interconnect a stem component insertable in bone (e.g., stem component 26 of FIGS. 1-2) and an articulation component adapted to mate with an opposed articular surface or an adjacent bone (e.g., articulation component 28 of FIGS. 1-2).

The component 132 can include a cavity 140 dimensioned to receive an adjacent component, such as another instance of the component 132 and/or one of the components 22 of the implant system 20. The cavity 140 may extend inwardly from the first end 136 of the main body 134 and may terminate prior to the second end 138 (see, e.g., FIG. 4).

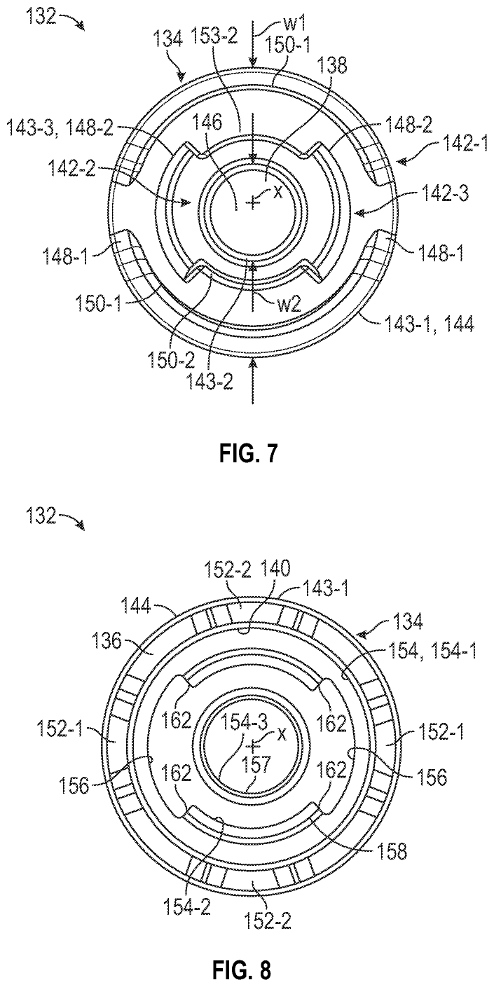

The first end 136 of the main body 134 may establish a first width W1, and the second end 138 may establish a second width W2, as illustrated in the implementation of FIG. 7. The widths W1, W2 may be established by a maximum distance across the respective first and second ends 136, 138. The first and second widths W1, W2 may be the same or may differ.

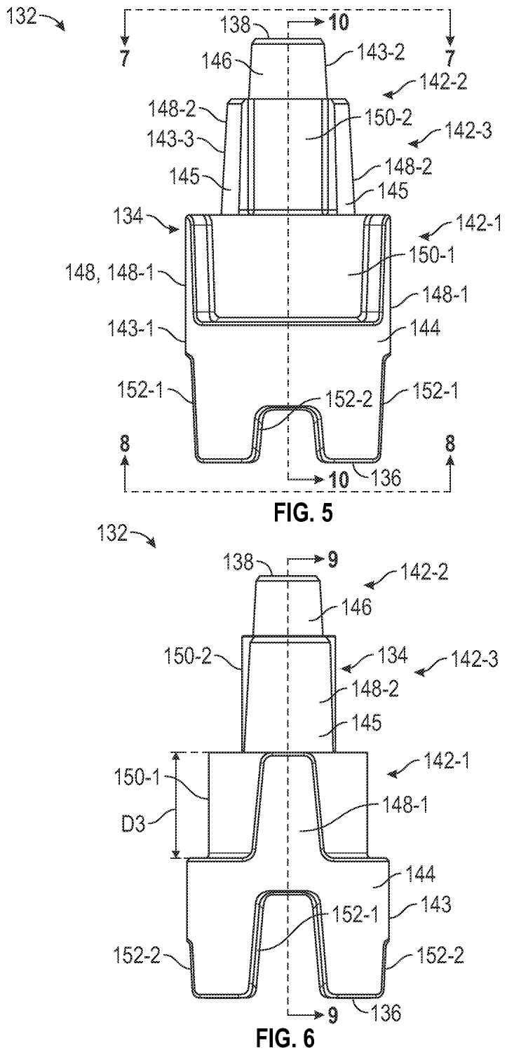

The main body 134 of the component 132 may have a generally stepped geometry established by a plurality of tiers 142 (see, e.g., FIGS. 5 and 6). The tiers 142 may be distributed along the axis X such that the first width W1 of the first end 136 may be greater than, or may otherwise differ from, the second width W2 of the second end 138 (see, e.g., FIG. 7). In implementations, the tiers 142 may be dimensioned such that the second width W2 may be less than or equal to approximately 50% of the first width W1, or more narrowly may be greater than or equal to approximately 25% of the first width W1.

The tiers 142 of the main body 134 may include at least a first (e.g., base or bottom) tier 142-1 and a second (e.g., top) tier 142-2. The tiers 142 may include at least one, or more than one, intermediate tier 142-3. The intermediate tier(s) 142-3 may be established between the first tier 142-1 and second tier 142-2. The first tier 142-1, second tier 142-2, and intermediate tier(s) 142-3 may be distributed along the axis X between the first end 136 and second end 138 of the main body 134. The intermediate tier(s) 142-3 may interconnect the first tier 142-1 and second tier 142-2. The stepped geometry of the main body 134 may be established such that the tiers 142 decrease in maximum width from the first tier 142-1 to the second tier 142-2. The component 132 may include a single intermediate tier 142-3. In implementations, the component 132 includes two or more intermediate tiers 142-3 differing in width to establish a stepped geometry of the main body 134. In other implementations, the intermediate tier(s) 142-3 may be omitted, and the second tier 142-2 may extend directly from the first tier 142-1.

The first tier 142-1 of the main body 134 may include a circumferential wall 144 extending circumferentially about the axis X. The circumferential wall 144 may establish the first end 136. The circumferential wall 144 may have a substantially cylindrical geometry (see, e.g., FIG. 7). For the purposes of this disclosure, the terms “about,” “approximately” and “substantially” mean±10 percent of the stated value or relationship unless otherwise indicated.

The second tier 142-2 of the main body 134 may include a protrusion 146. The protrusion 146 may establish the second end 138 of the main body 134. In implementations, the protrusion 146 may be a tapered protrusion that terminates at the second end 138.

Each of the tiers 142 of the main body 134 may have a respective periphery 143. The first tier 142-1 may have a first periphery 143-1. The first periphery 143-1 may be established by the circumferential wall 144. The second tier 142-2 may have a second periphery 143-2. The second periphery 143-2 may be established the protrusion 146. Each intermediate tier 142-3 may have an intermediate periphery 143-3. Each of the peripheries 143 may have various geometries and may have various surface features for establishing configurations of an assembly incorporating one or more instances of the component 132, such as the assembly 24 of FIGS. 1-2.

The main body 134 may include at least one or more abutments 148. The abutments 148 may be established along the periphery 143 of the main body 134 of the component 132. In implementations, the main body 134 may include a first (e.g., base) set of abutments 148-1 and/or a second (e.g., intermediate) set of abutments 148-2.

The first and second sets of abutments 148-1, 148-2 may be arranged relative to each other for establishing various configurations of an assembly incorporating one or more instances of the component 132, such as the assembly 24 of FIGS. 1-2. The first set of abutments 148-1 may be circumferentially distributed about the axis X along one of the tiers 142 of the component 132. The second set of abutments 148-2 may be circumferentially distributed about the axis X along another tier 142 of the component 132 such that the first set of abutments 148-1 are at least partially axially offset from the second set of abutments 148-2 relative to the axis X. The first set of abutments 148-1 may be at least partially circumferentially aligned with respective abutments 148 of the second set of abutments 148-2 relative to the axis X (see, e.g., FIGS. 5-7 and 9). In other implementations, the first set of abutments 148-1 may be circumferentially offset from the second set of abutments 148-2 relative to the axis X.

The first tier 142-1 may include the first set of abutments 148-1. The first periphery 143-1 may be established by the first set of abutments 148-1. The first set of abutments 148-1 may extend axially from the circumferential wall 144 in a direction towards the second end 138. In implementations, the first set of abutments 148-1 may be dimensioned to terminate at the intermediate tier 142-3. The abutments 148-1 may be dimensioned to taper in width in a direction towards the intermediate tier 142-3 and/or second end 138 relative to the axis X (see, e.g., FIG. 6).

One or more of the abutments 148 may be spaced apart from both the first end 136 and second end 138, such as the second set of abutments 148-2. The intermediate periphery 143-3 may be established by the second set of abutments 148-2. In implementations, the second tier 142-2 may include the second set of abutments 148-2.

Each of the first set of abutments 148-1 may have an arcuate face dimensioned to follow a substantially cylindrical profile (see, e.g., FIG. 7). The second set of abutments 148-2 may extend from an arcuate base dimensioned to follow a substantially circular profile (see, e.g., FIG. 7).

The periphery 143 of the main body 134 may include one or more cutouts 150. The cutouts 150 may be established between adjacent pairs of the abutments 148. In implementations, the cutouts 150 may include a first set of cutouts 150-1 and a second set of cutouts 150-2. The first set of cutouts 150-1 may be interspersed with the first set of abutments 148-1 about the axis X. The second set of cutouts 150-2 may be interspersed with the second set of abutments 148-2 about the axis X. The first set of cutouts 150-1 may be at least partially circumferentially aligned with the second set of cutouts 150-2 relative to the axis X (see, e.g., FIG. 7).

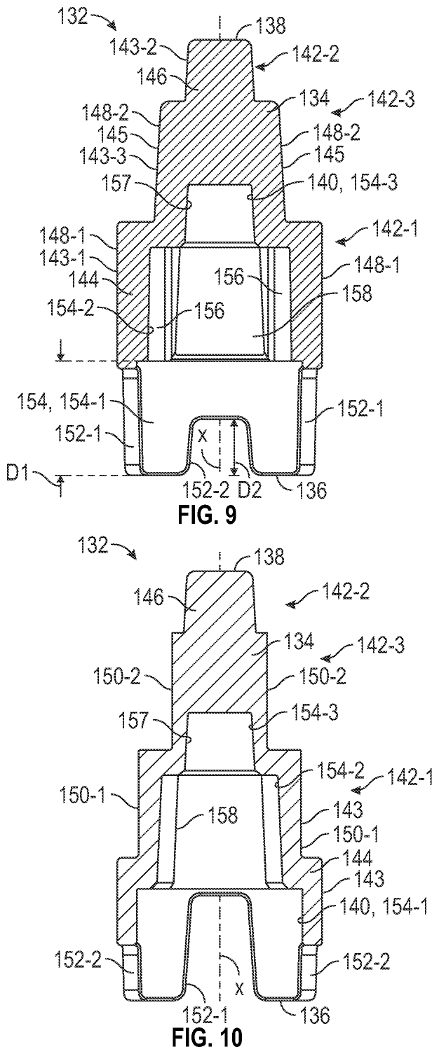

The intermediate periphery 143-3 may be dimensioned to taper or slope between the first tier 142-1 and the second tier 142-2 along circumferential faces 145 of the second set of abutments 148-2 (see, e.g., FIGS. 6 and 9). A width between the circumferential faces 145 of the second set of abutments 148-2 may be dimensioned to taper in a direction from the first tier 142-1 towards the second tier 142-2 and/or second end 138 relative to the axis X (see, e.g., FIGS. 5 and 9). In implementations, the intermediate periphery 143-2 may be substantially cylindrical.

The component 132 may include one or more recesses 152. The recesses 152 may be dimensioned to cooperate with another (e.g., adjacent) instance of the component 132 to establish various configurations of the assembly, such as the assembly 24. The recesses 152 may be circumferentially distributed about the axis X. The recesses 152 may be established by one or more of the tiers 142 of the main body 134. In implementations, the circumferential wall 144 may include one or more of the recesses 152. The recesses 152 may extend axially from the first end 136 in a direction towards the second end 138 relative to the axis X. The recesses 152 may be dimensioned to terminate prior to an axial position of the abutments 148 relative to the axis X.

A dimension of two or more recesses 152 may differ from each other to establish two or more distinct configurations of an assembly incorporating one or more of the components 132, such as the assembly 24. One or more of the recesses 152 may differ in length or height from at least one or more other recesses 152 relative to the axis X to establish a span of the configured assembly.

The recesses 152 may include at least one (e.g., collapsed or reduced) recess 152-1 and at least one (e.g., extended or heightened) recess 152-2. The collapsed recess 152-1 can be partially axially aligned and/or circumferentially offset from the extended recess 152-2 of the set of recesses 152 relative to the axis X (e.g., FIG. 8). In implementations, the component 132 may include a first set of collapsed recesses 152-1 and a second set of extended recesses 152-2. The collapsed recesses 152-1 may be interspersed with the extended recesses 152-2 relative to the axis X. The first and second sets of recesses 152-1, 152-2 may be established along a common tier 142. The first and/or second sets of recesses 152-1, 152-2 may be established along the circumferential wall 144 and may extend inwardly from the first end 136 of the main body 134. The abutments 148-1 may be axially spaced apart from the set of recesses 152-1, 152-2 relative to the axis X (see, e.g., FIGS. 5-6).

The component 132 may have various quantities of the recesses 152 to engage the abutments 148. A total quantity of the recesses 152 may be a multiple of a total quantity of the first set of abutments 148-1. In implementations, the total quantity of the recesses 152 is at least twice the total quantity of the first set of abutments 148-1. A total quantity of the first set of recesses 152-1 and/or a total quantity of the second set of recesses 152-2 may be equal to the total quantity of the first set of abutments 148-1.

The abutments 148 may taper in width a direction towards the second end 138 (see, e.g., FIG. 6). The tapering of the abutments 148-1 may facilitate insertion of the abutments 148-1 into the respective recesses 152.

The recesses 152 may have various geometries to complement one or more of the abutments 148. Each recess 152 of the first set of collapsed recesses 152-1 may have a first contour dimensioned to complement a profile of the first set of abutments 148-1. Each recess 152 of the second set of extended recesses 152-2 may have a second contour dimensioned to complement a profile of the first set of abutments 148-1. The second contour of the extended recesses 152-2 may differ from the first contour of the collapsed recesses 152-1. In implementations, the second contour of the extended recesses 152-2 may be dimensioned to complement a lesser portion of the profile of the first set of abutments 148-1 than the first contour of the collapsed recesses 152-1 such that the component 132 may sit at different heights on another component 132. The second profile of the extended recesses 152-2 may be dimensioned to substantially correspond to a lesser (e.g., terminal) portion of the first profile of the collapsed recesses 152-1. In the implementation of FIG. 9, the collapsed recesses 152-1 may extend a first distance D1, and the extended recesses 152 may extend a second distance D2 relative to the axis X. The first set of abutments 148-1 may be dimensioned to extend a third distance D3 relative to the axis X (FIG. 6). The first distance D1 of the collapsed recesses 152-1 may be substantially equal to or less than the third distance D3 of the first set of abutments 148-1 relative to the axis X. The second distance D2 may be less than the first distance D1 relative to the axis X such that the second set of recesses 152-2 may only receive a lesser portion of the first set of abutments 148-1 than the first set of recesses 152-1. In implementations, the second distance D2 may be less than or equal to approximately 75% of the first distance D1, or more narrowly greater than or equal to approximately 25% of the first distance D1. In implementations, the second distance D2 may be approximately 50% of the first distance D1. The second distance D2 may be at least 5% of a total length of the component 132 between the first and second ends 136, 138. The different distances D1, D2 of the recesses 152-1, 152-2 may be utilized to establish a distinct set of configurations of adjacent components 132 in an installed position.

The recesses 152 may be established relative to the position of the abutments 148. In implementations, the first set of collapsed recesses 152-1 may be at least partially circumferentially aligned with the first set of abutments 148-1 relative to the axis X (e.g., FIG. 6). The second set of extended recesses 152-2 may be at least partially circumferentially aligned with the first set of cutouts 150-1 relative to the axis X (e.g., FIG. 5). The first set of abutments 148-1 may be at least partially circumferentially aligned with the set of collapsed recesses 152-1, but may be circumferentially offset from the set of extended recesses 152-2 relative to the axis X (see, e.g., FIGS. 5 and 6).

Referring to FIG. 8, with continuing reference to FIG. 4, the cavity 140 can be dimensioned to cooperate with a profile of an exterior or periphery 143 of another instance of the component 132 insertable into the cavity 140. In implementations, a profile of the cavity 140 may be dimensioned to cooperate with a profile of at least the first tier 142-1, second tier 142-2 and/or the intermediate tier(s) 142-3 of the main body 134 of another instance of the component 132 insertable into the cavity 140.

The cavity 140 may have various geometries to cooperate with an adjacent instance of the component 132. The cavity 140 may include one or more cavity levels (e.g., tiers) 154 distributed along the axis X. The cavity levels 154 may include a first cavity level 154-1, second cavity level 154-2 and/or third cavity level 154-3 distributed along the axis X. In other implementations, the component 132 may include fewer or more than a total of three cavity levels 154, such as only one or two cavity levels 154.

The cavity 140 may be dimensioned such that the cavity levels 154 establish a generally stepped geometry decreasing in maximum width. The cavity levels 154 may decrease in maximum width from the first cavity level 154-1 to the third cavity level 154-3 (e.g., FIGS. 8-10).

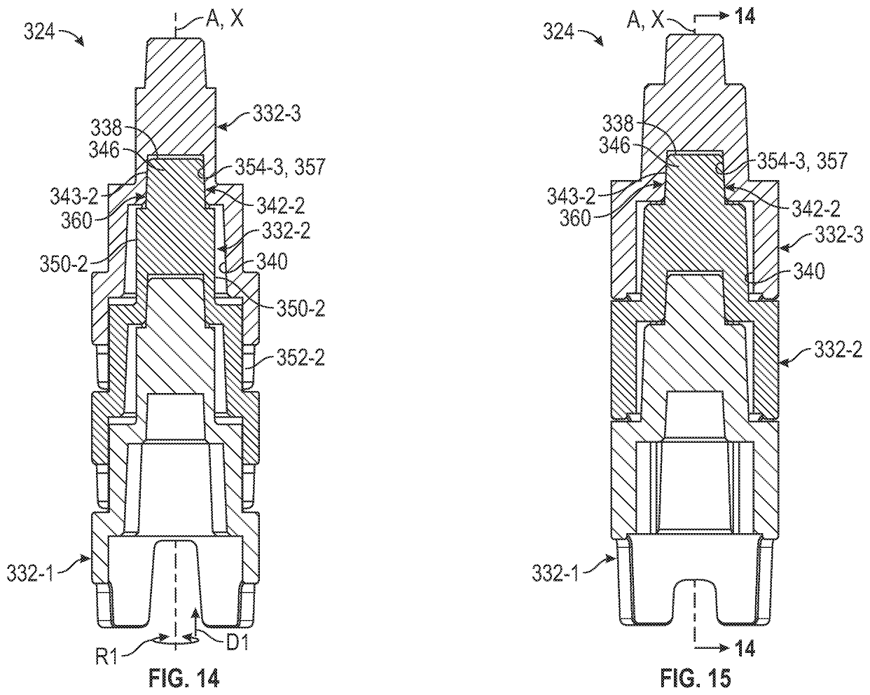

The cavity levels 154 may be dimensioned to complement one or more features of the component 132. A surface of the third cavity level 154-3 may be dimensioned to mate with a surface of the protrusion 146 of another instance of the component 132 to establish an interference fit for opposing relative movement. The third cavity level 154-3 may have a third tapered cavity periphery 157 (e.g., FIGS. 8-10). In implementations, a surface of the third tapered cavity periphery 157 of the third cavity level 154-3 may be dimensioned to cooperate with, and complement, the profile of the tapered protrusion 146 of the second tier 142-2 of another instance of the component 132 to establish a first Morse taper connection (see, e.g., tapered protrusion 346 and Morse taper connection 360 of FIGS. 14-15).

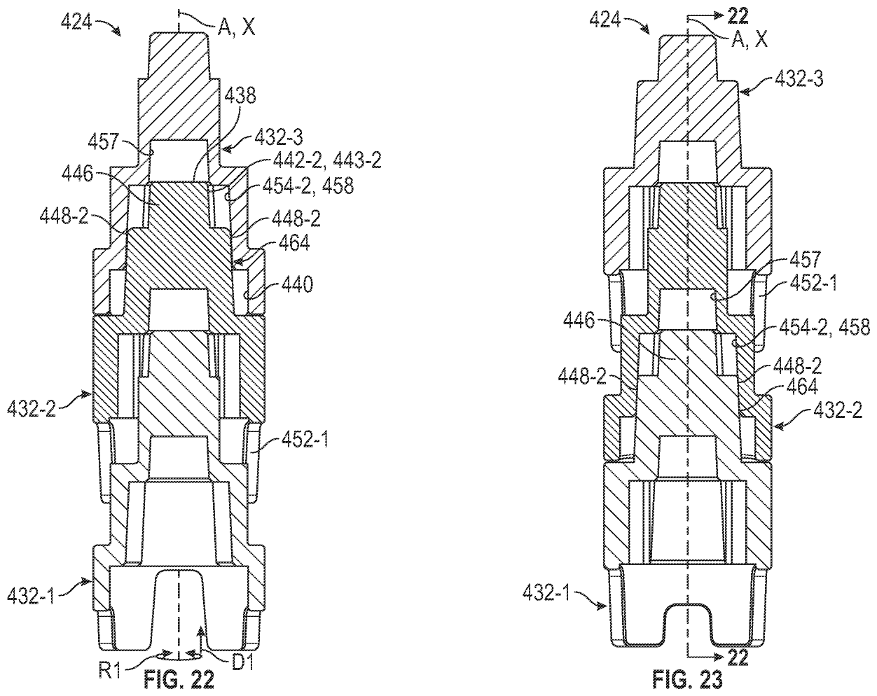

A surface of the second cavity level 154-2 may be dimensioned to mate with a surface of the second set of abutments 148-2 of another instance of the component 132 to establish an interference fit for opposing relative movement. The second cavity level 154-2 may have a second cavity periphery 158 (e.g., FIGS. 8 and 10). The second cavity periphery 158 may be tapered in width. In implementations, a surface of the second cavity periphery 158 of the second cavity level 154-2 may be dimensioned to cooperate with, and complement, the profile of the second set of abutments 148-2 of another instance of the component 132 to establish a second Morse taper connection (see, e.g., abutments 448-2 and Morse taper connection 464 of FIGS. 22-23).

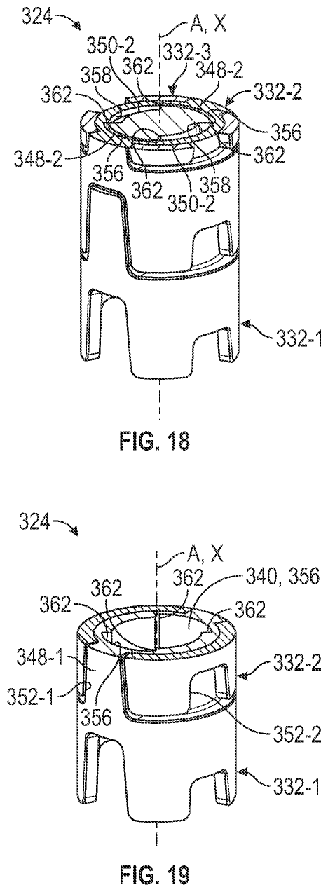

The cavity 140 may include a set of cavity cutouts 156. Circumferentially opposed engagement faces 162 may bound the respective cavity cutouts 156 (FIG. 4). The engagement faces 162 may be dimensioned to engage the second set of abutments 148-2 of another instance of the component 132. The cavity cutouts 156 and second set of abutments 148-2 of another instance of the component 132 may cooperate to establish a keyed interface to limit relative rotation between the components 132. The cavity cutouts 156 may be circumferentially distributed about the axis X. In implementations, the set of cavity cutouts 156 may be established along the second cavity level 154-2. The cavity cutouts 156 may be dimensioned to extend radially outwardly from the second cavity periphery 158 of the second cavity level 154-2 relative to the axis X (see also FIG. 4). A total number of the cavity cutouts 156 may be greater than or equal to a total number of the second set of abutments 148-2. The set of cavity cutouts 156 may be dimensioned to mate with, and complement, a profile of the second set of abutments 148-2 of another instance of the component 132 to limit relative rotation (see, e.g., abutments 348-2 of FIGS. 17-18).

FIG. 11 illustrates an assembly 224 that may be utilized to establish a portion of an implant system 220, such as the implant system 20. The assembly 224 may be established by a set of components 232. Each of the components 232 may be dimensioned according to the component 132 of FIGS. 3-10. Each component 232 may include one or more abutments 248 and one or more recesses 252. The abutments 248 may include a first set of abutments 248-1 and a second set of abutments 248-2. The recesses 252 may include a first set of (e.g., collapsed) recesses 252-1 and a second set of (e.g., extended) recesses 252-2.

The set of components 232 may be configured or arranged in a stacked relationship along an assembly axis A of the assembly 224. The components 232 may be dimensioned such that (e.g., longitudinal) axes X of the components 232 may be substantially aligned along the assembly axis A in an installed position. In the implementation of FIG. 11, the set of components 232 may include a first component 232-1, a second component 232-2 and a third component 232-3. It should understood that fewer or more than a total of three components 232 may be utilized to establish the assembly 224 in accordance with the teachings disclosed herein. The components 232 may be provided as a kit to a surgeon for establishing the implant system 200, which may be incorporated into the implant system 20 of FIGS. 1 and 2.

Each pair of adjacent components 232 may be configurable to establish a first (e.g., collapsed) configuration and/or a second (e.g., extended) configuration of a respective portion of the assembly 224. The collapsed configuration may establish a first span of the adjacent pair of components 232. The extended configuration may establish a second, relatively greater span of the adjacent pair of components 232. In the configuration of FIG. 11, the first component 232-1 and second component 232-2 are arranged in the collapsed configuration, and the second component 232-2 and third component 232-3 are arranged in the extended configuration. The adjacent pair of the components 232-1, 232-2 and the adjacent pair of components of 232-2, 232-3 may establish respective portions of the assembly 224.

The collapsed configuration may be established in response to inserting the second end 238 of the adjacent component 232 into the cavity 240 of the component 232 (see, e.g., second end 338 of FIG. 14) and inserting at least one or more abutments 248, such as the first set of abutments 248-1, of the adjacent component 232 into a respective one of the collapsed recesses 252-1. The extended configuration may be established in response to inserting the second end 238 of the adjacent component 232 into the cavity 240 (see, e.g., second end 438 of FIG. 22) and inserting at least one or more of the abutments 248, such as the first set of abutments 248-1, of the adjacent component 232 into a respective one of the extended recesses 252-2. The components 232 may be dimensioned such that the first set of abutments 248-1 are insertable into only a selected one of the set of collapsed recesses 252-1 or the set of extended recesses 252-2, but not simultaneously into both the first and second sets of recesses 252-1, 252-2, to establish the respective collapsed configuration or extended configuration of the adjacent pair of components 232.

Insertion of the first set of abutments 248-1 into a selected one of the collapsed recesses 252-1 and the extended recesses 252-2 may establish a keyed interface, which may limit relative rotation and facilitate alignment between the components 232.

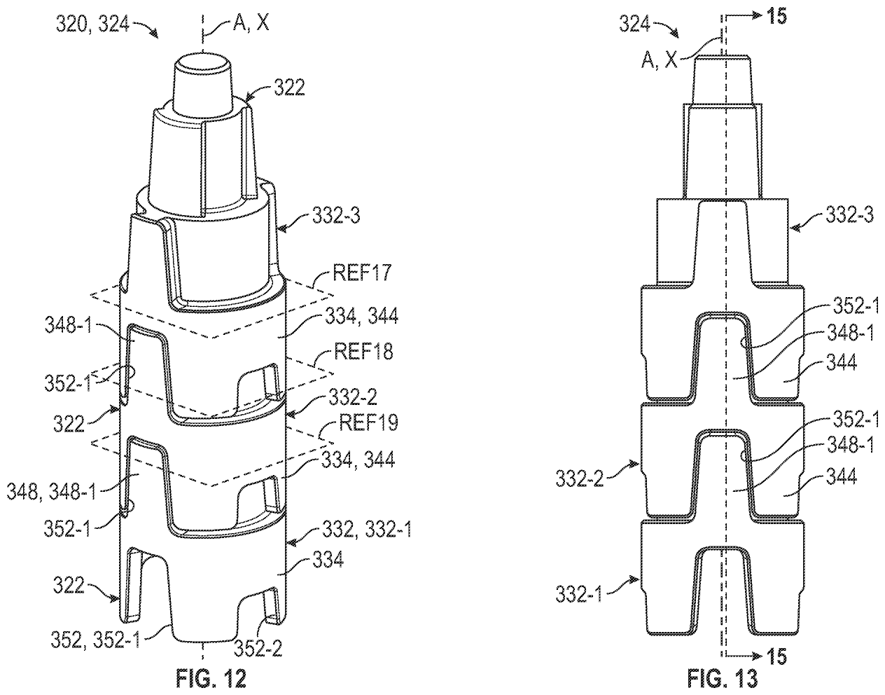

FIGS. 12-19 illustrate another configuration of an assembly 324 established by a set of components 332. The set of components 332 can include a first component 332-1, second component 332-2 and third component 332-3. Each of the components 332 can be dimensioned according to any of the components disclosed herein, such as the component 132. The components 332 of the assembly 324 may be the same components 232 of the assembly 224 of FIG. 11, but may have one or more pairs of the components 332 arranged in a configuration that differs from a configuration of a respective pair of the components 232 of the assembly 224. The components 232 of FIG. 11 may be reconfigured to establish the assembly 324 and vice versa. Each adjacent pair of the components 332 may be configurable or arranged to establish at least a first (e.g., collapsed) configuration or second (e.g., extended) configuration of a respective portion of the assembly 324. In the configuration of FIGS. 12-19, each adjacent pair of components 332 is arranged to establish a collapsed configuration of a respective portion of the assembly 324.

Each component 332 may include a plurality of abutments 348. The abutments 348 may include a first set of abutments 348-1 and a second set of abutments 348-2. Each component 332 may include a plurality of recesses 352. The recesses 352 may include a first set of (e.g., collapsed) recesses 352-1 and a second set of (e.g., extended) recesses 352-2.

Each abutment 348 of the first set of abutments 348-1 of the component 332-2/332-3 may be insertable into a selected one of the recesses 352 of the adjacent component 332-1/332-2, such as one of the collapsed recesses 352-1, to establish a collapsed configuration of the adjacent pair of components 332-1/332-2, 332-2/332-3. A main body 334 of the component 332 may be dimensioned such that surfaces bounding the collapsed recess 352-1 sit on the respective abutment 348-1 in response to inserting the abutment 348-1 into the collapsed recess 352-1 to establish the collapsed configuration. A circumferential wall 344 of the component 332-1/332-2 establishing the collapsed recess 352-1 may be dimensioned to straddle the respective abutment 348-1 in the collapsed configuration to limit relative rotation between the components 332-1/332-2, 332-2/332-3 with respect to the assembly axis A.

Referring to FIGS. 14-15, with continuing reference to FIGS. 12-13, a second end 338 of an adjacent one of the components 332 may be inserted into the cavity 340 of the component 332 and may be positioned to establish an interference fit to limit relative movement between the components 332. A third cavity level 354-3 of the cavity 340 of the component 332-1/332-2 may be dimensioned to cooperate with a tapered protrusion 346 of a second tier 342-2 of the adjacent component 332-2/332-3 in the collapsed configuration to establish a first Morse taper connection 360. The component 332-1/332-2 may be impacted onto the tapered protrusion 346 of the component 332-2/332-3 to establish the Morse taper connection 360. The Morse taper connection 360 may oppose relative axial and/or rotational movement between the components 332. The third cavity level 354-3 of the component 332-1/332-2 may be dimensioned to cooperate with, and complement, the tapered protrusion 346 of the adjacent component 332-2/332-3 in the collapsed configuration, but not in the extended configuration (see, e.g., tapered protrusion 446 of FIGS. 22-23 and 25), to establish the respective first Morse taper connection 360.

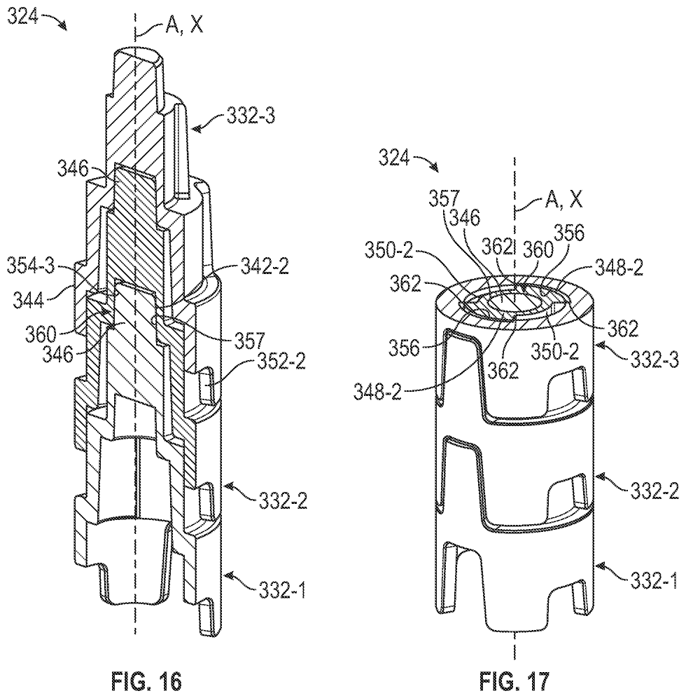

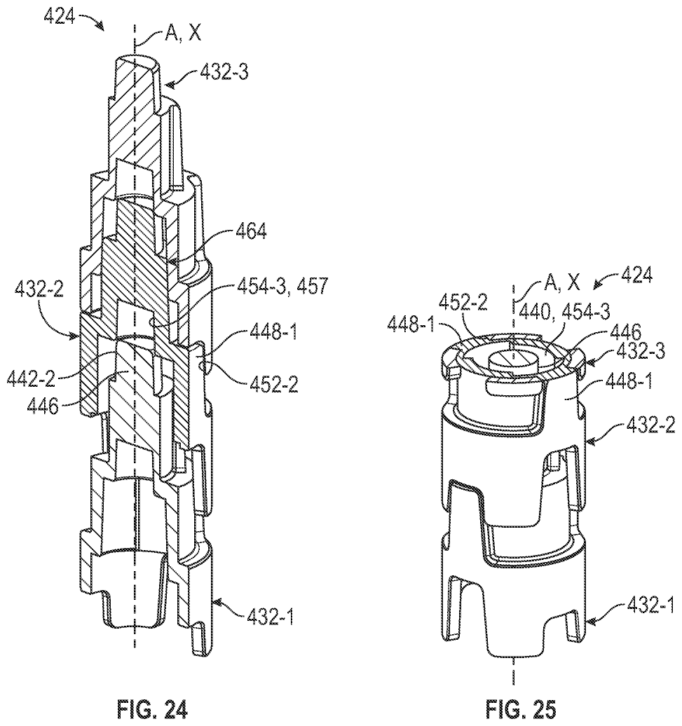

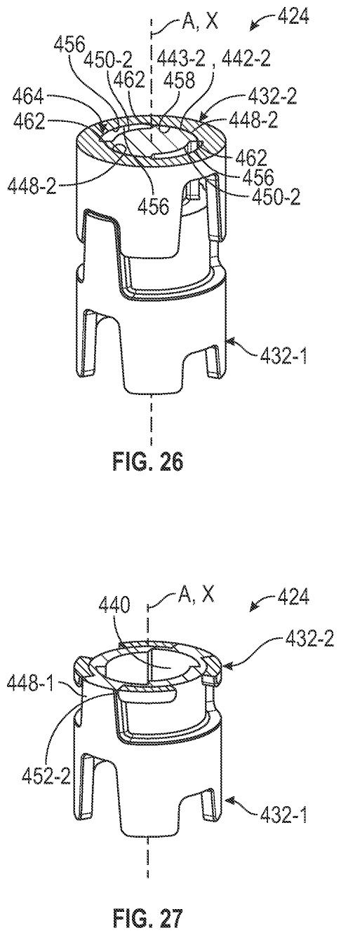

Referring to FIGS. 17 and 18, with continuing reference to FIGS. 12-16, the components 332 may include a set of cavity cutouts 356 along the cavity 340. The set of cavity cutouts 356 may be dimensioned to serve as a keyway and the second set of abutments 348-2 may serve as a set of keys insertable into the cavity cutouts 356 to establish a keyed interface such that the tapered protrusion 346 may pass through the second cavity level 354-2 and at least partially into the third cavity level 354-3 to engage surfaces of the third cavity level 354-3. Engagement between surfaces of the tapered protrusion 346 and a third tapered cavity periphery 357 of the third cavity level 354-3 may establish a first Morse taper connection 360 (FIGS. 16 and 17). The cavity 340 may have a tapered cavity periphery 358 along the second cavity level 352-2 (FIG. 18). The tapered cavity periphery 358 may be dimensioned to block movement of the tapered protrusion 346 through the second cavity level 354-2 and into the third cavity level 354-3 such that the tapered protrusion 346 may be disengaged from the third tapered cavity periphery 357 (see, e.g., tapered protrusion 456 of FIGS. 24-25 and tapered cavity periphery 458 of FIG. 26). The tapered cavity periphery 358 may be dimensioned to block engagement between the tapered protrusion 346 and the third tapered cavity periphery 357 in the extended configuration of the components 332. A set of cutouts 350-2 between the second set of abutments 348-2 may complement, but may be disengageable from, the tapered cavity periphery 358 in the collapsed configuration such that the tapered protrusion 346 may pass through the second cavity level 354-2 and at least partially into the third cavity level 354-3 to establish the Morse taper connection 360 (see, e.g., FIG. 18).

The set of cavity cutouts 356 of the component 332-1/332-2 may be dimensioned to mate with the second set of abutments 348-2 of the adjacent components 332-2/332-3 in the collapsed configuration, but not in the extended configuration (see, e.g., cavity cutouts 456 of FIG. 26), to establish the keyed interface to limit relative rotation with respect to the assembly axis A. Circumferentially opposed engagement faces 362 may bound the respective cavity cutouts 356 (see also FIG. 4). The engagement faces 362 of the component 332-1/332-2 may be dimensioned to limit relative rotation between the adjacent components 332-1/332-2, 332-2/332-3 in the collapsed configuration, but not in the extended configuration (see, e.g., engagement faces 462 of FIG. 26), in response to engagement between the second set of abutments 348-2 and the respective engagement faces 362 relative to the assembly axis A. The engagement faces 362 may facilitate alignment of the adjacent components 332 during assembly.

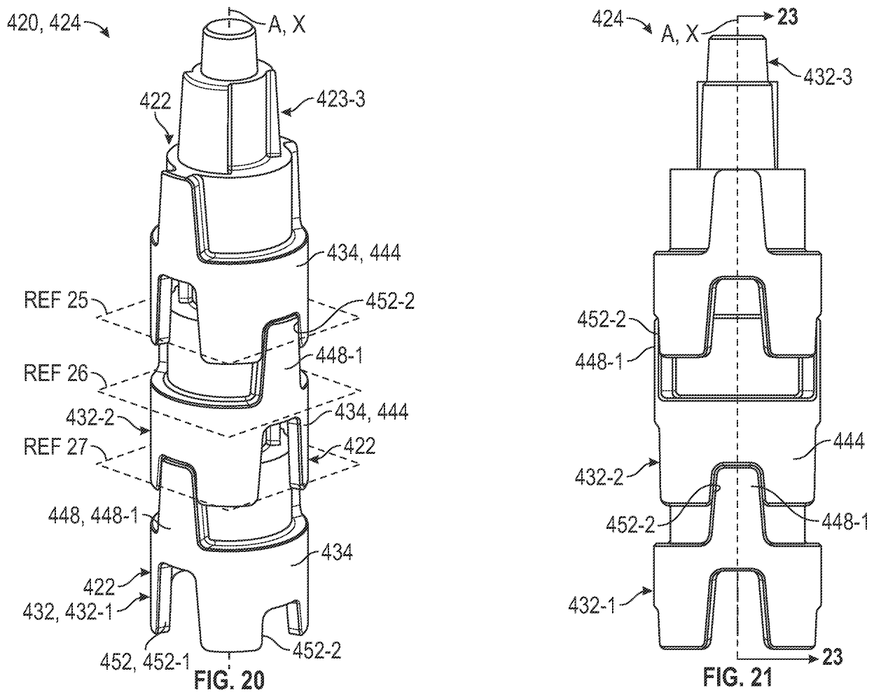

FIGS. 20-27 illustrate another configuration of an assembly 424 established by a set of components 432. The set of components 432 may include a first component 432-1, second component 432-2 and third component 432-3. Each of the components 432 can be dimensioned according to any of the components disclosed herein, such as the component 132. The components 432 of the assembly 424 may be the same components 432 of the assembly 224 of FIG. 11 and/or the assembly 324 of FIGS. 12-19, but may have one or more pairs of the components 432 arranged in a configuration that differs from a configuration of a respective pair of the components 232/332 of the assembly 224/324. The components 232 of FIG. 11 and/or components 332 of FIGS. 12-19 may be reconfigured to establish the assembly 424 and vice versa. In the implementation of FIGS. 20-27, the assembly 424 is configured such that each adjacent pair of the components 432 is configured in the extended configuration to establish respective portions of the assembly 424.

The configuration of the assembly 324 of FIGS. 12-19 may establish a minimum configured height or length of a common set of components 232/332/432. The configuration of the assembly 424 of FIGS. 20-27 may establish a maximum configured height or length of the common set of components 232/332/432. The configuration of the assembly 224 of FIG. 11 may establish configured height or length of the common set of components 232/332/432 that is greater than the maximum configured height or length of the assembly 324 but that is less than the maximum configured height or length of the assembly 424. The common set of components 232/332/432 can be selected by the surgeon or assistant and configured intraoperatively and/or preoperatively according to a surgical plan. Although the common set of the components 232/332/432 are illustrated as having a total of three of the components 232/332/432, it should be understood that fewer or more than three of the components 232/332/432 may be provided and/or selected for establishing the assembly 224/324/424.

Each component 432 may include a plurality of abutments 448 including a first set of abutments 448-1 and a second set of abutments 448-2. Each component 432 may include a plurality of recesses 452 including a first set of (e.g., collapsed) recesses 452-1 and a second set of (e.g., extended) recesses 452-2.

Each abutment 448 of the first set of abutments 448-1 of the component 432-2/432-3 may be insertable into a selected one of the recesses 452 of the adjacent component 432-1/432-2, such as one of the extended recesses 452-2, to establish an extended configuration of the adjacent pair of components 432-1/432-2, 432-2/432-3. The main body 434 may be dimensioned such that surfaces bounding the extended recess 452-2 sit on the respective abutment 448-1 in response to inserting the abutment 448-1 into the extended recess 452-2 to establish the extended configuration. A circumferential wall 444 of the component 432-1/432-2 establishing the extended recess 452-2 may be dimensioned to straddle the respective abutment 448-1 in the extended configuration to limit relative rotation between the components 432-1/432-2, 432-2/432-3 with respect to assembly axis A.

Referring to FIGS. 22-25, with continuing reference to FIGS. 20-21, the second end 438 of an adjacent one of the components 432 may be inserted into the cavity 440 of the component 432. The adjacent component 432 may be positioned in the cavity 440 to establish an interference fit to limit relative movement between the components 432. The cavity 440 may have a tapered cavity periphery 458 along the second cavity level 452-2. The tapered cavity periphery 458 may be dimensioned to cooperate with the second set of abutments 448-2 or another portion of the tapered periphery 343-2 of the second tier 342-2 in the extended configuration (see, e.g., FIGS. 23 and 26), but not in the collapsed configuration (see, e.g., abutments 348-2 and tapered cavity periphery 358 of FIG. 18), to establish a second Morse taper connection 464. The Morse taper connection 464 may oppose relative axial and/or rotational movement between the components 432. The component 432-1/432-2 may be impacted onto the second set of abutments 448-2 of the component 432-2/432-3 to establish the respective second Morse taper connection 464.

In the extended configuration of the components 432-2, 432-3 of FIGS. 24-25, the tapered protrusion 446 of the component 432-3 is disengaged or otherwise spaced apart from a surface of the third cavity level 454-3 of the adjacent component 432-2. In the extended configuration of the adjacent components 432-2, 432-3 of FIG. 26, the cavity cutouts 456 and engagement faces 462 of the component 432-2 are circumferentially offset from the second set of abutments 448-2 of the component 432-3.

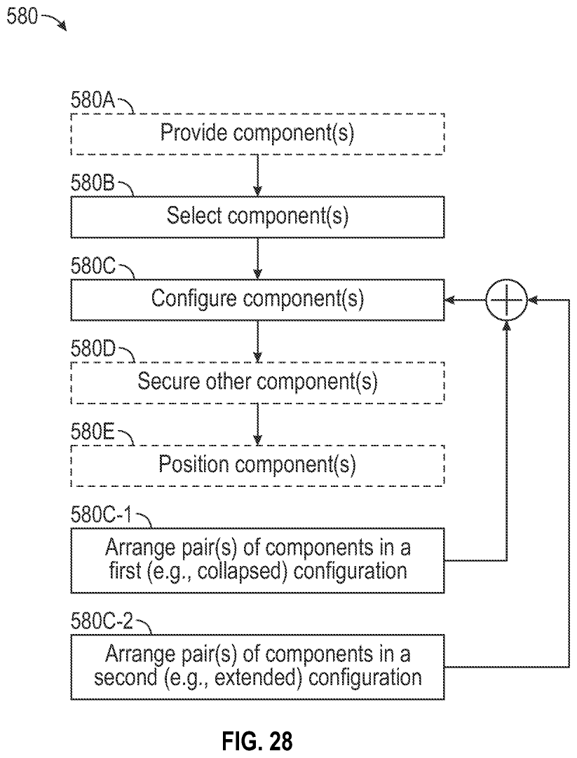

FIG. 28 illustrates a method of installing an orthopaedic implant system in flow chart 580 according to an implementation. The method 580 may be utilized to perform an arthroplasty to restore functionality to a bone and/or joint, including any of the bones and joints disclosed herein. In implementations, the method 580 may be utilized in repair of a fractured proximal humerus or other long bone. The method 580 may be utilized in an anatomical shoulder or reverse shoulder arthroplasty. The method 580 can be utilized in accordance with any of the components and implant systems disclosed herein, including the components 22, 122, 222, 322, 422 of the implant systems 20, 220, 320, 420, such as the components 32, 132, 232, 332, 432. Reference is made to the implant systems 20, 220, 320, 420 for illustrative purposes. Fewer or additional steps than are recited below could be performed within the scope of this disclosure, and the recited order of steps is not intended to limit this disclosure.

At step 580A, one or more components may be provided to the surgeon or assistant. The components may include any of the components disclosed herein, including the components 22, 122, 222, 322, 422. The components may be provided to the surgeon as a kit. The kit may include components of various shapes, sizes and quantities. The components may be selected from the kit to establish a specified configuration of the implant system according to an anatomy of the patient and other aspects of a surgical plan.

At step 580B, the surgeon or assistant may select one or more components from the set of components. The set of components may be predetermined according to a surgical plan such that the surgeon or assistant selects every component of the set. The surgeon or assistant may select fewer than every component of the set and/or may remove one or more of the components preoperatively and/or intraoperatively during configuration of the respective implant system.

Referring to FIG. 11, with continuing reference to FIG. 28, step 580B may include selecting at least three components 222 from the set of components 222, such as the components 232-1, 232-2, 232-3. Step 580B may include selecting one or more other components of the set, such as the stem component 22, articulation component 28 and/or head component 30 of FIG. 1.

At step 580C, the surgeon or assistant may configure the selected components to establish the implant system. The components 222 may include one or more components 232 configurable to establish an assembly 224. Each adjacent pair of the components 232 may be arranged in at least two or more distinct configurations, including at least a first (e.g., collapsed) configuration and/or a second (e.g., extended) configuration. Each adjacent pair of the components 232 may be configurable to establish at least a first (e.g., collapsed) configuration and a second (e.g., extended) configuration of a respective portion of the assembly 224. Step 380C may include configuring or arranging each adjacent pair of the selected components 232-1/232-2, 232-2/232-3 in either the collapsed configuration at step 580C-1 or the extended configuration at step 580C-2 to establish the respective portion of the assembly 224.

Adjacent pairs of the components may be arranged in the same configuration (see, e.g., FIGS. 12 and 20) or may be arranged in a different configuration than one or more adjacent pairs of the components (see, e.g., FIG. 11). Step 580C may include configuring an adjacent pair of the selected components 232-1, 232-2 in the collapsed configuration, but configuring another adjacent pair of the selected components 232-2, 232-3 in the extended configuration to establish the respective portions of the assembly 224, as illustrated by the assembly 224 of FIG. 11. In the configuration of the assembly 224, the component 232-2 may be common to two adjacent pairs of the components 232-1/232-2, 232-2/232-3.

Various techniques may be utilized to establish the configurations of the selected components, including any of the techniques and arrangements disclosed herein. Referring to FIGS. 14 and 22, with continuing reference to FIG. 28, step 580C may include inserting the second end 338/438 of the adjacent component 332/432 into the cavity 340/440. Inserting the second end 338/438 of the adjacent component 332/432 into the cavity 340/440 may include moving the adjacent component 332/432 in a direction D1. The direction D1 may be substantially parallel to the axis X of the component 332/432. Referring to FIGS. 17 and 26, with continuing reference to FIG. 28, step 380C may occur such that a wall of the cavity 340/440 engages the second set of abutments 348-2/448-2 of the adjacent component 332/432 to oppose relative rotation between the components 332/432 with respect to the assembly axis A. The surfaces of the components 332/432 may engage each other utilizing any of the techniques disclosed herein to the establish the collapsed and extended configurations.

Referring to FIGS. 14-17, with continuing reference to FIG. 28, inserting the second end 338 of the adjacent component 332 into the cavity 340 at step 580C may occur such that surfaces of the third cavity level 354-3 cooperate with the tapered periphery 343-2 of the second tier 342-2 of the adjacent component 332 in the collapsed configuration, but not in the extended configuration (see, e.g., second tier 442-2 of FIG. 22), to establish the Morse taper connection 360. Step 580C-1 may include impacting the component 332-2/332-3 onto the tapered protrusion 346 of the component 332-1/332-2 to establish the Morse taper connection 360.

Referring to FIG. 17, with reference to continuing reference to FIGS. 14-16 and 28, the collapsed configuration may be established at step 580C-1 in response to inserting the second set of abutments 348-2 of the adjacent component 332-2/322-3 into the set of cavity cutouts 356 of the component 332-1/332-2 to establish a keyed interface and then engaging the periphery 357 of the third cavity level 354-3 with the third tapered periphery 343-3 of the adjacent component 332-2/322-3 to establish the first Morse taper connection 360. Step 580C-1 may occur such that the first Morse taper connection 360 is established, but not the second Morse taper connection between the tapered cavity periphery 358 and the second set of abutments 348-2 (see, e.g., second Morse taper connection 464 of FIG. 26). Inserting the second end 338 of the adjacent component 332 into the cavity 340 may include inserting the second set of abutments 348-2 into the set of cavity cutouts 356 in the collapsed configuration, but not in the extended configuration (see, e.g., cavity cutouts 456 of FIG. 26), such that circumferentially opposed engagement faces 362 bounding the respective cavity cutouts 356 limit relative rotation between the adjacent components 332 with respect to the assembly axis A.

Referring to FIGS. 12-13, with continuing reference to FIG. 28, the collapsed configuration may be established in response to inserting at least one abutment 348 of an adjacent component 332-2/332-3 into a selected recess 352 of the component 332-1/332-2, such as one of the collapsed recesses 352-1. Step 380C-1 may include inserting the first set of abutments 348-1 of the adjacent component 332-2/332-3 into the collapsed recesses 352-1 of the component 332-1/332-2 to establish the collapsed configuration. The extended configuration may be established in response to inserting the abutment 348 of the adjacent component 332-2/332-3 into another selected recess 352 of the component 332-1/332-2, such as one of the extended recesses 352-2. Step 380C may include rotating the components 332/432 relative to each other to align the first set of abutments 348-1/448-1 with the selected set of collapsed recesses 352-1/452-1 and extended recesses 352-1/452-2 prior to the insertion (see, e.g., rotational direction R1 of FIGS. 14 and 22).

Referring to FIG. 26, with reference to FIGS. 22-24 and 28, the extended configuration may be established at step 380C-2 in response to engagement between the tapered periphery 458 of the second cavity level 454-2 of the component 432-1/432-2 and the second set of abutments 448-2 of the adjacent component 432-2/432-3 to establish the second Morse taper connection 464. The extended configuration may be established such that the second Morse taper connection 464 may block engagement between the third tapered cavity periphery 457 of the third cavity level 454-3 of the component 432-1/432-2 and the third tapered periphery 443-3 of the adjacent component 432-2/432-3 (see, e.g., FIGS. 22-24).

Referring to FIGS. 20-21, with continuing reference to FIG. 28, step 380C-2 may include inserting the set of abutments 448-1 of the adjacent component 432-2/432-3 into the extended recesses 452-2 of the component 432-1/432-2 to establish the extended configuration. Referring to FIGS. 22-24 and 26, with continuing reference to FIG. 28, inserting the second end 438 of the adjacent component 432 into the cavity 440 at step 580C may occur such that surfaces of the second cavity level 454-2 cooperate with the second set of abutments 448-2 of the adjacent component 432 in the extended configuration at step 580C-2, but not in the collapsed configuration at step 580C-1 (see, e.g., abutments 348-2 of FIG. 18), to establish the Morse taper connection 464. Step 580C-2 may include impacting the component 432-2 onto the second set of abutments 448-2 of the component 432-3 to establish the Morse taper connection 464.

Referring to FIG. 1, with continuing reference to FIG. 28, one or more other components 22 may be secured to the components 32 configured at step 580C. Step 580D may include securing one of the components 32 of the assembly 24 to a stem component 26.

At step 580E, the components 22 may be positioned in the patient prior to, during and/or subsequent to steps 580C and 580D. The position and/or orientation of the components 22 may specified in a surgical plan or may be determined intraoperatively. Step 580E may include inserting the stem component 26 into bone B (shown in dashed lines in FIG. 2). In implementations, step 580E may include inserting one or more of the components 32 of the assembly 24 into the bone B.

Step 580E may include securing another one of the selected components 32 of the assembly 24 to the articulation component 28. The articulation component 28 may be adapted to mate with an opposed articular surface AS of an adjacent implant or an adjacent bone to restore functionality to a joint (see, e.g., FIG. 2). Method 580 can include one or more additional steps performed prior to, during and/or subsequent to steps 580A-580E. Method 580 may include positioning one or more bone fragments and/or soft tissue about the components 32 and/or another portion of the implant system 20.

The novel implant systems and methods of this disclosure may provide versatility in repairing bone defects restoring functionality to bones and/or joints. The components may be provided to the surgeon as a kit. The surgeon may select one or more of the components from the kit and may configure the components to establish an assembly incorporated into the implant system. The assembly may be configured according to a specified dimension of the implant system, which may be determined preoperatively or intraoperatively. The components in the kit utilized to establish the assembly may be identical, which may reduce complexity of the system, or may differ in geometry. The components may be arranged in two or more configurations, such as a collapsed configuration and an extended configuration, in response to engagement between features along the component and another adjacent component. The features may be used to establish one or more tapered connections and/or keyed interfaces to limit relative movement between the adjacent components, which may improve stability and alignment between the components and improve healing of the patient.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure.

What is claimed is:

1. A component for an orthopaedic procedure comprising:
a main body extending along a longitudinal axis between a first end and a second end, the main body implantable in tissue, and the main body including a cavity extending inwardly from the first end;
wherein the main body has a stepped geometry established by a plurality of tiers including a first tier having a first tapered periphery and a second tier having a second tapered periphery, and the second tapered periphery is established by circumferential faces of a first set of abutments distributed about the longitudinal axis; and
wherein the cavity includes a first cavity level and a second cavity level distributed along the longitudinal axis, the first cavity level includes a periphery dimensioned to complement a profile of the first tapered periphery, the second cavity level includes a periphery dimensioned to complement a profile of the second tapered periphery, a set of cavity cutouts extend outwardly from the periphery of the second cavity level, and the set of cavity cutouts are dimensioned to complement a profile of the first set of abutments.

2. The component as recited in claim 1, wherein:
the first tier includes a tapered protrusion establishing the first tapered periphery, and the tapered protrusion extends to the second end.

3. The component as recited in claim 1, wherein:
the plurality of tiers includes a third tier having a periphery established by a circumferential wall and a second set of abutments extending axially from the circumferential wall towards the second end, and the second set of abutments are distributed about the longitudinal axis; and
wherein the circumferential wall includes first and second sets of recesses extending from the first end, the first set of recesses are interspersed with the second set of recesses, the first set of recesses have a first contour dimensioned to complement a profile of the second set of abutments, and the second set of recesses have a second, different contour dimensioned to complement a lesser portion of the profile of the second set of abutments.

4. The component as recited in claim 3, wherein:
the first set of recesses extend a first distance, and the second set of recesses extend a second distance that is less than the first distance relative to the longitudinal axis;
a first set of cutouts are interspersed with the first set of abutments about the longitudinal axis; and
the first set of recesses are at least partially circumferentially aligned with the second set of abutments, and the second set of recesses are at least partially circumferentially aligned with the first set of cutouts relative to the longitudinal axis.

5. The component as recited in claim 4, wherein the first set of abutments are at least partially circumferentially aligned with the second set of abutments relative to the longitudinal axis.

6. The component as recited in claim 1, wherein the main body is dimensioned to interconnect a stem component insertable in bone and an articulation component adapted to mate with an opposed articular surface of an adjacent implant or an adjacent bone.

7. An orthopaedic implant system comprising:
a set of components stackable to establish an assembly, each of the components comprising:
a main body extending along an axis between a first end and a second end, and a cavity extending inwardly from the first end;
wherein the main body has a stepped geometry established by a plurality of tiers distributed along the axis, the plurality of tiers include a first tier having a first tapered periphery and a second tier having a second tapered periphery, and the second tapered periphery is established by a first set of abutments; and
wherein the cavity includes a first cavity level and a second cavity level distributed along the axis, a set of cavity cutouts extend outwardly from a periphery of the second cavity level;
wherein each adjacent pair of the components is configurable to establish a collapsed configuration and an extended configuration of a respective portion of the assembly, the collapsed configuration is established in response to inserting the first set of abutments into the set of cavity cutouts to establish a keyed interface such that a first Morse taper connection is established between a periphery of the first cavity level and the first tapered periphery, but the extended configuration is established in response to engagement between the periphery of the second cavity level to establish a second Morse taper connection that blocks engagement between the periphery of the first cavity level and the first tapered periphery.

8. The system as recited in claim 7, wherein:
the first tapered periphery of the first tier is established by a tapered protrusion terminating at the second end.

9. The system as recited in claim 7, wherein:
circumferentially opposed engagement faces bounding the respective cavity cutouts are dimensioned to limit relative rotation between the adjacent components in the collapsed configuration, but not in the extended configuration, in response to engagement between the first set of abutments and the respective engagement faces.

10. The system as recited in claim 7, wherein:
the plurality of tiers includes a third tier, the third tier includes a second set of abutments circumferentially distributed about the axis and a circumferential wall having a plurality of recesses extending from the first end, and the plurality of recesses include a set of extended recesses interspersed with a set of collapsed recesses; and
the collapsed configuration is established in response inserting the second set of abutments of the adjacent component into the set of collapsed recesses, but the extended configuration is established in response to inserting the second set of abutments of the adjacent component into the set of extended recess.

11. The system as recited in claim 10, wherein:
the set of collapsed recesses have a first contour dimensioned to complement a profile of the second set of abutments, and the set of extended recesses have a second, different contour dimensioned to complement a lesser portion of the profile of the second set of abutments.

12. The system as recited in claim 10, wherein the second set of abutments are at least partially circumferentially aligned with the set of collapsed recesses, but are circumferentially offset from the set of extended recesses relative to the axis.

13. The system as recited in claim 7, further comprising:
a stem component insertable into bone;
an articulation component adapted to mate with an opposed articular surface of an adjacent implant or an adjacent bone; and
wherein the assembly interconnects the stem component and the articulation component in an installed position.

14. A method of installing an orthopaedic implant system, comprising:
selecting components from a set of components;
wherein each of the components comprises a main body having a stepped geometry established by a plurality of tiers distributed along an axis between a first end and a second end, the plurality of tiers including a first tier having a first tapered periphery and a second tier having a second tapered periphery, the second tapered periphery established by a first set of abutments, and the main body including a cavity having a first cavity level and a second cavity level distributed along the axis, and a set of cavity cutouts extend outwardly from a periphery of the second cavity level;
wherein each adjacent pair of the components are configurable to establish a collapsed configuration and an extended configuration of a respective portion of an assembly;
wherein the collapsed configuration is established in response inserting the first set of abutments of the adjacent component into the set of cavity cutouts to establish a keyed interface and then engaging a periphery of the first cavity level with the first tapered periphery of the adjacent component to establish a first Morse taper connection, but the extended configuration is established in response to engagement between the periphery of the second cavity level and the first set of abutments of the adjacent component to establish a second Morse taper connection; and
configuring each adjacent pair of the selected components in the collapsed configuration or the extended configuration to establish the respective portion of the assembly.

15. The method as recited in claim 14, wherein the extended configuration is established such that the second Morse taper connection blocks engagement between the periphery of the first cavity level and the first tapered periphery of the adjacent component.

16. The method as recited in claim 15, wherein the step of establishing the keyed interface occurs such that circumferentially opposed engagement faces bounding the respective cavity cutouts limit relative rotation between the adjacent components with respect to the axis.

17. The method as recited in claim 14, wherein:
the plurality of tiers includes a third tier, the second tier interconnects the first and third tiers, the third tier includes at least one abutment established along a periphery of the main body, and the main body includes at least one extended recess and at least one collapsed recess extending from the first end; and
the collapsed configuration is established in response to inserting the at least one abutment of the adjacent component into the at least one collapsed recess, but the extended configuration is established in response to inserting the at least one abutment of the adjacent component into the at least one extended recess.

18. The method as recited in claim 17, wherein the first set of abutments are at least partially circumferentially aligned with a second set of abutments relative to the axis.

19. The method as recited in claim 14, wherein:
the selecting step includes selecting at least three of the components from the set of components; and
the configuring step includes configuring an adjacent pair of the selected components in the collapsed configuration but configuring another adjacent pair of the selected components in the extended configuration to establish the respective portions of the assembly.

20. The method as recited in claim 14, further comprising:
securing one of the selected components of the assembly to a stem component;
inserting the stem component into bone; and
securing another one of the selected components of the assembly to an articulation component, wherein the articulation component is adapted to mate with an opposed articular surface of an adjacent implant or an adjacent bone.
